(19) United States
(12) Patent Application Publication
(10) Pub. No.: US 2021/0106601 A1
(43) Pub. Date: Apr. 15, 2021

(54) MANGIFERIN AS A PROTECTIVE AGENT AGAINST MITOCHONDRIAL DNA DAMAGE AND SKIN-CARE COMPOSITIONS COMPRISING SAME

(71) Applicant: Olusola Clement IDOWU, Gateshead (GB)

(72) Inventor: Olusola Clement IDOWU, Gateshead (GB)

(21) Appl. No.: 16/965,022

(22) PCT Filed: Jan. 29, 2019
(86) PCT No.: PCT/GB2019/050234

§ 371 Date: Jul. 27, 2020

(30) Foreign Application Priority Data
Jan. 30, 2018 (GB) 1801475.3
Jul. 6, 2018 (GB) 1811143.5

(51) Int. Cl. A61K 31/7048; C07H 7/06; A61K 8/60; A61K 8/73; A61K 9/00; A61P 17/16; A61Q 19/00; A61K 9/06; A61K 9/107; A61Q 17/04; A61K 8/06; A61K 47/40; A61K 47/69; C08B 37/16; A61K 8/9789; A61K 36/22; A61K 8/40; A61K 8/35; A61K 8/29

(52) U.S. Cl. CPC A61K 31/7048; A61K 8/29; A61K 8/602; A61K 8/738; A61K 9/0014; A61P 17/16; A61Q 19/00; A61K 9/06; A61K 9/107; A61Q 17/04; A61K 8/062; A61K 47/40; A61K 47/6951; C08B 37/0015; A61K 8/9789; A61K 36/22; A61K 8/40; A61K 8/35; C07H 7/06

(57) ABSTRACT

The invention relates to compounds that provide protective effects against mitochondrial DNA damage. In particular, it relates to mangiferin, ideally in the form of a naturally-occurring mangiferin extract, and to mangiferin-containing compositions and their use as protective agents against mitochondrial DNA damage. Aspects of the invention relate to an additive for a skin care composition, wherein the additive comprises a complex of mangiferin with β-cyclodextrin or a β-cyclodextrin derivative. The invention also relates to skin care compositions comprising the mangiferin complexes, as well as to methods of their preparation. In embodiments, the additive can be used in a sunscreen formulation to protect against UV-induced mtDNA damage.

### TECHNICAL FIELD

The present invention relates to compounds that provide protective effects against mitochondrial DNA damage. In particular, it relates to mangiferin, ideally in the form of a naturally-occurring mangiferin extract or one of its isomers or derivatives, and mangiferin-containing compositions and their use as protective agents against mitochondrial DNA damage. In aspects, the invention relates to an additive for a skin care composition, wherein the additive comprises a complex of mangiferin with β-cyclodextrin or a β-cyclodextrin derivative. The invention also relates to skin care compositions comprising the complexes, as well as to methods of the preparation of the complexes. Further aspects of the invention relate to use of the skin care compositions to protect against UV-induced mitochondrial DNA damage.

### BACKGROUND

The mammalian mitochondrial genome is a circular molecule present in multiple (often 2-10) copies in each mitochondrion, with hundreds to thousands of mitochondria per cell (more mitochondria are generally present in cells with high energy requirements). The human mitochondrial (mtDNA) encodes 2 rRNAs, 22 tRNAs, and 13 polypeptides, all of which are involved in oxidative phosphorylation through the electron transport chain (ETC). While the role of nuclear DNA (nDNA) damage in human pathological conditions such as cancers is well known, increasing attention is being focused on the association between mitochondrial DNA (mtDNA) damage and various human diseases.

Substantial evidence suggests that mtDNA may be more vulnerable than nDNA to certain kinds of damage, in particular reactive oxygen species (ROS)-mediated lesions and oxidative stress. Oxidative DNA damage to cells is an unavoidable consequence of cellular metabolism; however, interactions with exogenous sources such as carcinogenic compounds, redox-cycling drugs, ionising and UV radiation also contribute to oxidative damage. Mitochondrial damage can be an indicator of oxidative stress. mtDNA damage can be triggered by free radicals produced directly from cells in oxidative stress and from environmental aggressors such as genotoxic chemicals and environmental pollutants. mtDNA damage can be triggered by energy radiation from sources such as UVA, UVB and UVC radiation and, in addition to cancer, has been implicated in the manifestation of various age-related disorders and degenerative diseases in humans, such as skin ageing, melanoma neuro-degeneration, cancer, stroke, cardiomyopathy and diabetes.

The skin is the largest organ in the human body, and performs protective, regulatory and sensory functions. Its primary role, however, is to act as a protective barrier against external forces, such as UV radiation, to which it is typically exposed on a daily basis. UV absorption through the skin generates oxygen-derived free radicals, which can induce DNA damage via the production of a range of photoproducts. This process can change the base pairing abilities of normal DNA, resulting in mutations. These mutations can disrupt tumour suppressor genes such as P53 and INK4A, leading to an increased risk of cancer. Skin disease in particular represents a major health care challenge in today's world. With more than one million new cases of skin cancer diagnosed each year in the United States alone (National Cancer Institute, www.cancer.gov), reducing the risk factors for skin cancer is a significant concern.

Conventionally, sunscreen or sun-cream compositions contain additives that protect against UV damage by preventing UV rays from penetrating the skin. Nowadays, however, due to the heightened awareness of the links between DNA damage caused by UV radiation and such age-related and degenerative disorders as discussed above, the use of UV-filtering additives has spread increasingly to many other cosmetic items, such as daily moisturisers and fake tan products, in an effort to protect against DNA damage and to confer anti-aging properties on the product for cosmetic reasons. These products can be formulated differently to suncreams, and often have different formulation requirements (such as formulation consistency, colour etc.). There is additionally increasing consumer-led demand for organic products, or those containing organic or natural ingredients.

UV filtering additives typically function either physically (i.e. by reflecting UV radiation) or chemically (i.e. by absorbing UV radiation), to prevent UV radiation reaching the skin. Commonly used physical UV filtering additives include titanium dioxide (TiO₂) and zinc oxide (ZnO). These function by forming an opaque layer over the skin, which reflects the UV light in both the UVA and UVB spectra. However, these additives typically result in a characteristic white colour, with the resulting formulations exhibiting poor sensorial feel and poor absorption into the skin. While consumers may accept these formulation limitations in a suncream, they tend to be less acceptable in other cosmetic products where the primary focus is not necessarily UV protection. Increasing evidence also points to mtDNA damage arising from the use of TiO₂ and ZnO (“Oxidative stress-induced cytotoxic and genotoxic effects of nano-sized titanium dioxide particles in human HaCaT keratinocytes”; Jaeger A et al; Toxicology, vol. 296(1-3), (2012): pp. 27-36; “DNA damaging potential of zinc oxide nanoparticles in human epidermal cells”; Sharma V et al. Toxicol. Lett., Vol. 185(3), (2009): pp. 211-218; and “Mechanism of oxidative DNA damage induced by quercetin in the presence of Cu(II)”; Yamashita N et al. Mutat. Res., Vol. 425(1), (1999): pp. 107-115).

Chemical UV filtering additives can address some of these downsides; however no single active agent provides high enough protection in both the UVA and UVB spectra, requiring that they be used in combination. This can cause formulation issues, due to photoinstability and inter-reaction of the UVA and UVB filtering components. In addition, most chemical UV filtering additives offer poor UV protection in the UVA spectrum (315 to 400 nm).

Consequently, identifying non-toxic skincare compositions with the requisite UV absorption properties, but which do not lead to mtDNA damage, can be extremely difficult.

It is an object of the present invention to mitigate or obviate one or more of the disadvantages associated with the prior art. In particular, it would be advantageous to provide compounds for use as protective agents against mitochondrial DNA damage. It would also be advantageous to provide an additive for a skin care composition comprising high levels of UV protection, and/or which could prevent mitochondrial DNA damage caused by UV rays. An organic composition, and/or one which avoids or reduces the use of inorganic particles such as titanium dioxide and zinc oxide would be particularly beneficial, as would an organic composition with improved formulation characteristics, such as improved sensorial feel and/or improved skin absorbance. In addition, a method of preparing such a composition, which methods provides certain advantages such as ease of manufacture, reduction in cost, reduction in waste etc. would be useful.

### SUMMARY OF THE INVENTION

Mangiferin is a xhanthone, mainly found in higher plants. It was first isolated from mango leaves (as C2-beta-D-glucopyranosil-1,3,6,7-tethraydroxanthone), and later from mango bark (as 1,6,7-trihydroxy-3-methoxy-2-C-beta-D-glucopyranosyl-xanthone), where the resultant content of mangiferin was reported to be higher.

Mangiferin may be obtained for example by purifying extracts of all or part of these plants using any extraction or purification process (e.g. extraction with a polar solvent such as water, an alkanol, or mixture of these solvents, subsequent purification by crystallization or any other method known to those skilled in the art.

Previously, mangiferin has been associated with a variety of potential pharmacological effects including antimicrobial activities. However, the inventors have surprisingly found that mangiferin has protective effects against mitochondrial DNA damage. Notably, it is not necessarily the case that an antimicrobial or antioxidant composition would provide protective effect against mitochondrial DNA damage. In fact, it is known that antioxidants may also cause direct DNA damage to human cells, suggesting that antioxidant activity alone is not sufficient to suggest suitability for use in skin care or cosmetic products; for instance, epigallocatechin gallate (EGCG), an exemplary antioxidant found in green tea, was found to induce significant DNA damage in human cells [“Antioxidant Induces DNA Damage, Cell Death and Mutagenicity in Human Lung and Skin Normal Cells”: Linda Y. Lu, Ning Ou & Qing-Bin Lu; www.nature.com Scientific Reports 3; Article 3169 (2013)]. Many antimicrobials and antioxidants are known for their generation of ROS and to cause mtDNA damage in mammalian cells [“Bactericidal Antibiotics Induce Mitochondrial Dysfunction and Oxidative Damage in Mammalian Cells”; Sameer Kalghatgi et al; Sci Trans!. Med. 5(192): 2013]. Thus, the finding that mangiferin has protective effects against mitochondrial DNA damage is surprising and unexpectedly advantageous.

According to an aspect of the present invention there is provided a topical composition comprising a compound of formula I:

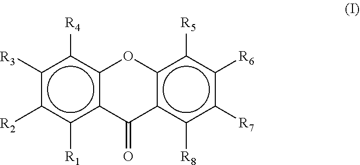

wherein each of R₁, R₂, R₃, R₄, R₅, R₆, R₇, R₈ is selected from the group consisting of —H, —OH and a glucosyl radical, or an ester or pharmaceutically acceptable salt thereof, for the prevention of mitochondrial DNA damage.

In an embodiment, the compound of formula I is mangiferin, having the formula:

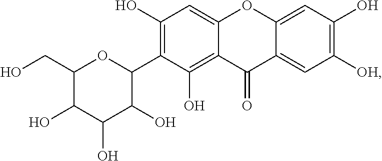 [SMILES: O=c1c2cc(O)c(O)cc2oc2cc(O)c(C3OC(CO)C(O)C(O)C3O)c(O)c12]

or isomers or derivatives or an ester thereof, or one of its pharmaceutically acceptable salts.

According to an aspect of the invention there is provided an additive for a skin care composition, wherein the additive comprises a complex of mangiferin with β-cyclodextrin or a 62 -cyclodextrin derivative.

The invention also relates to methods of preparation of such additives and compositions. Complexation of the mangiferin with β-cyclodextrin or a β-cyclodextrin derivative increases availability of the mangiferin in aqueous solution whilst retaining its high absorbance and mtDNA protection characteristics. Accordingly, aspects of the invention relate to the use of mangiferin-containing skin care compositions to protect against UV-induced mtDNA damage.

According to an aspect of the invention there is provided a method for preventing mitochondrial DNA damage in mammals, the method comprising the steps of: administering a topical composition comprising a compound of formula I:

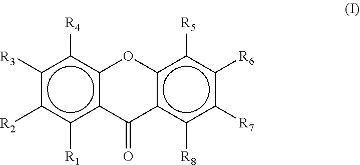

wherein each of R₁, R₂, R₃, R₄, R₅, R₆, R₇, R₈ is selected from the group consisting of —H, —OH and a glucosyl radical, or of one of the esters or its pharmaceutically acceptable salts for the prevention of mitochondrial DNA damage.

The compound may be mangiferin, having the following formula:

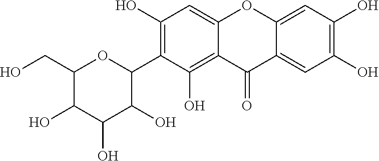 [SMILES: O=c1c2cc(O)c(O)cc2oc2cc(O)c(C3OC(CO)C(O)C(O)C3O)c(O)c12]

or isomers or derivatives or an ester thereof, or one of its pharmaceutically acceptable salts.

Various further features and aspects of the invention are defined in the claims.

### BRIEF DESCRIPTION OF THE DRAWINGS

Embodiments of the present invention will now be described by way of example only with reference to the accompanying drawings where like parts are provided with corresponding reference numerals and in which:

FIG. 1 shows the structure of mangiferin;

FIG. 2 shows viability assays for a range of mangiferin extract concentrations;

FIG. 3 shows MtDNA damage in primary human skin fibroblasts and human skin cell line (HDFn) cells following UV irradiation;

FIG. 4 shows mtDNA damage in human skin cell line (HDFn) cells following UV irradiation in the presence of mangiferin;

FIG. 5 shows mtDNA damage in human skin cell line (HDFn) cells following UV irradiation in the presence of commercial sun screen products (SPF0, SPF15 and SPF30);

FIG. 6 is a UV absorbance scan for mangiferin at 1000 μg/ml;

FIG. 7 shows UV absorbance scans for mangiferin-βcyclodextrin complexes;

FIG. 8 shows basal respiration of a mangiferin-containing sunscreen formulation and control formulations (water-based formulation, leading commercial product formulation) as measured on the Seahorse analyser;

FIG. 9 shows mitochondrial respiration of a mangiferin-containing sunscreen formulation and control formulations (water-based formulation, leading commercial product formulation) as measured on the Seahorse analyser;

FIG. 10 shows glycolytic index of a mangiferin-containing sunscreen formulation and control formulations (water-based formulation, leading commercial product formulation) as measured on the Seahorse analyser.

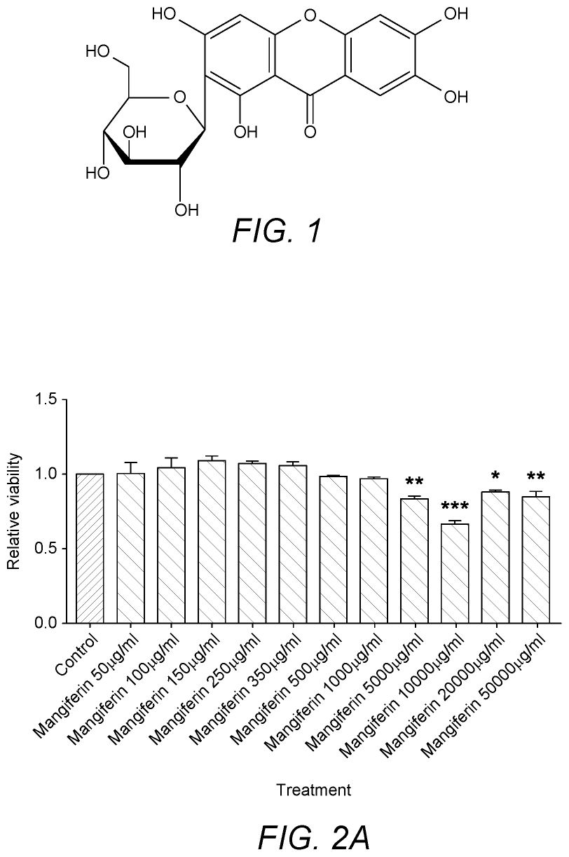

### DETAILED DESCRIPTION

The present invention relates to a topical composition comprising a compound of formula I:

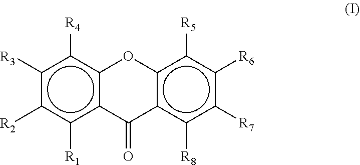

wherein each of R₁, R₂, R₃, R₄, R₅, R₆, R₇, R₈ is selected from the group consisting of —H, —OH and a glucosyl radical, or of one of the esters or its pharmaceutically acceptable salts, for the prevention of mitochondrial DNA damage.

In an embodiment, the compound of formula I is mangiferin, having the formula:

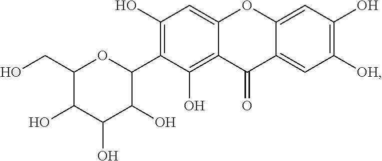 [SMILES: O=c1c2cc(O)c(O)cc2oc2cc(O)c(C3OC(CO)C(O)C(O)C3O)c(O)c12]

or isomers or derivatives or an ester thereof, or one of its pharmaceutically acceptable salts.

The compound may be a naturally-occurring plant extract.

The compound may be a naturally-occurring mangiferin extract.

Optionally, the mangiferin extract is present at an effective concentration for protecting against DNA damage.

Optionally, the mangiferin extract is present at an effective concentration of 1,000 μg/ml or greater.

Optionally, the mangiferin extract is present at an effective concentration of between 1,000 μg/ml and 5,000 μg/ml.

Optionally, the composition is a pharmaceutical composition.

Alternatively, the composition is a cosmetic composition.

The composition may be in the form of a cream, oil, ointment or lotion.

The invention also relates to the use of the compounds described above to protect the mitochondrial DNA of living cells from damage.

The damage may be from reactive oxygen species (ROS), oxidative stress, exposure to UVA and/or UVB etc.

Preferably the living cells are mammalian cells. More preferably the living cells are human skin cells.

According to an aspect of the present invention there is provided a method for preventing mitochondrial DNA damage in mammals, comprising the steps of: administering a topical composition comprising a compound having the following general formula I:

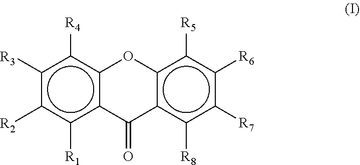

wherein each of R₁, R₂, R₃, R₄, R₅, R₆, R₇, R₈ is selected from the group consisting of —H, —OH and a glucosyl radical, or of one of the esters or its pharmaceutically acceptable salts.

Preferably the compound is mangiferin, having the formula:

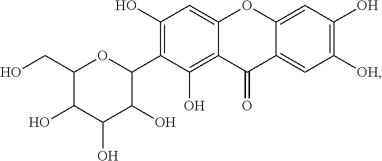 [SMILES: O=c1c2cc(O)c(O)cc2oc2cc(O)c(C3OC(CO)C(O)C(O)C3O)c(O)c12]

or isomers or derivatives or an ester thereof or one of its pharmaceutically acceptable salts.

Preferably the compound is a naturally occurring plant extract.

Most preferably the compound is naturally occurring mangiferin extract.

Optionally the mangiferin extract is present at an effective concentration for protecting against DNA damage.

Optionally the mangiferin extract is present at an effective concentration of 1000 μg/ml or greater.

Optionally the mangiferin extract is present at an effective concentration of between 1000 μg/ml and 5000 μg/ml.

Preferably the method is for preventing mitochondrial DNA damage in humans.

More preferably the method is for preventing mitochondrial DNA damage in human skin cells. Yet more preferably the method is for preventing mitochondrial DNA damage in human dermal fibroblast cells.

According to another aspect of the present invention there is provided an in vitro method of inhibiting mitochondrial DNA damage in living cells comprising providing a test sample of living cells and providing a protective composition between the test sample and source of potential damage, the protective composition comprising a compound having the following general formula I:

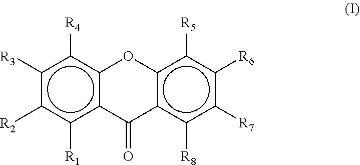

wherein each of R₁, R₂, R₃, R₄, R₅, R₆, R₇, R₈ is selected from the group consisting of —H, —OH and a glucosyl radical, or of one of the esters or its pharmaceutically acceptable salts, for the prevention of mitochondrial DNA damage.

Preferably the compound is mangiferin, having the formula:

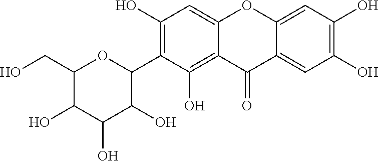 [SMILES: O=c1c2cc(O)c(O)cc2oc2cc(O)c(C3OC(CO)C(O)C(O)C3O)c(O)c12]

or isomers or derivatives or an ester thereof or one of its pharmaceutically acceptable salts.

Preferably the compound is a naturally occurring plant extract.

Most preferably the compound is naturally occurring mangiferin extract.

Optionally the mangiferin extract is present at an effective concentration for protecting against DNA damage.

Optionally the mangiferin extract is present at an effective concentration of 1000 μg/ml or greater.

Optionally the mangiferin extract is present at an effective concentration of between 1000 μg/ml and 5000 μg/ml.

Optionally the method comprises contacting said compound with said test sample.

The invention also relates to an additive for a skin care composition, wherein the additive is a complex of mangiferin with β-cyclodextrin or a β-cyclodextrin derivative.

Mangiferin (MGN) is a naturally-occurring xhantone, (1,3,6,7-tetrahydroxy-2-[3,4,5-trihydroxy-6-(hydroxymethyl)oxan-2-yl]xanthen-9-one) mainly found in higher plants such as Mangifera indica L (commonly known as mango). The inventors have advantageously determined that mangiferin shows good UV absorbance, while protecting against mtDNA damage. However, mangiferin has poor solubility in water, limiting its use, particularly in cosmetic products which often require aqueous compositions or phases.

The mangiferin can be a naturally-occurring mangiferin extract, or a synthetic analog thereof.

Advantageously, the additive complexes according to the invention show good solubility in water, and can be formulated into aqueous cosmetic compositions and into emulsions at functionally-relevant and economically-viable concentrations. Skin care compositions to which the additives can be incorporated include moisturisers, tinted moisturisers, primers, foundations, fake tan products, toners and serums, for example, as well as more specific sun-care products such as sunscreens.

According to the present invention, the additive comprises mangiferin complexed with β-cyclodextrin or a β-cyclodextrin derivative.

In an embodiment, the β-cyclodextrin derivative is hydroxypropyl-β-cyclodextrin, sulfo-butyl-ether β-cyclodextrin or mono (6-ethylene-diamino-6-deoxy)-β-cyclodextrin.

In an embodiment, the stoichiometric ratio of mangiferin:β-cyclodextrin/β-cyclodextrin derivative is 1:1.

The additive may be used for the prevention of mitochondrial DNA damage.

The additive may consist of the mangiferin/β-cyclodextrin or mangiferin/β-cyclodextrin derivative complex or a mixture of these, or may comprise additional components.

The invention also relates to a skin care composition, comprising the mangiferin complex additive described above. The additive can be included in the skin care composition at an effective concentration to protect against mtDNA damage. In an embodiment, the additive can be included in the skin care composition such that the concentration of the mangiferin is from 1 to 10% (w/w).

Advantageously, the inventors have determined that when the mangiferin-βcyclodextrin complex is incorporated into a formulation within this concentration range, good UV absorbance can be achieved, while the mtDNA damaging-preventing functionality of the mangiferin is retained.

The skin care composition may be a pharmaceutical skin care composition or may be a cosmetic skin care composition.

The composition may comprise at least one additional ingredient selected from an organic UV filter, an inorganic UV filter, an emulsifier, a suspending agent, a film former and a skin conditioner.

The skin care composition may be an aqueous composition.

Alternatively, the skin care composition may be an emulsion. The emulsion may comprise the mangiferin complex in an aqueous phase.

As would be understood by one skilled in the art, the term “emulsion” is intended to encompass oil-in-water emulsions as well as water-in-oil emulsions, and multiple emulsions (such as water-in-oil-in water etc.).

According to an aspect of the invention, there is provided a method of using the skin care compositions described above to protect against UV-induced mtDNA damage. Such methods involve topically administering the skin care compositions to the skin. In an embodiment, the skin is mammalian skin, such as human skin.

The skin care composition may be a topical composition.

The skin care composition may be in the form of a cream, oil, ointment, lotion, gel or spray.

The skin care composition may comprise additional UV protection or UV filtering additives which can work in conjunction with the additive of the invention to provide broad UV protection. Advantageously, mangiferin has been demonstrated to show good absorbance in the UVA spectrum, where traditionally, suncream ingredients have shown poor protection.

The invention also relates to a method of preparing an additive for a skin care composition, the method comprising:

preparing a solution of β-cyclodextrin or a β-cyclodextrin derivative in water;

adding mangiferin to the solution;

allowing the complex to form at a temperature between 30° C. and 50° C. for between 3 to 6 hours; and

removing solids by filtration.

Surprisingly, the inventors have determined that a longer reaction/complexation time, does not lead to any increase in solubility for the resultant complex. Accordingly, when the complexation reaction is allowed to take place over a 3 to 6 hour period, this increases the efficiency of the process, particularly from a commercial perspective.

In an embodiment, the step of preparing a solution of β-cyclodextrin or a β-cyclodextrin derivative in water comprises preparing a solution comprising β-cyclodextrin or a β-cyclodextrin derivative at approximately its solubility limit. In an embodiment, the β-cyclodextrin or a β-cyclodextrin derivative is added at a concentration of from 1 to 3% (w/w).

In an embodiment, the step of adding mangiferin to the solution comprises adding mangiferin at, or approximately at, its solubility limit in water. In an embodiment, mangiferin is added at a concentration of from 0.1-0.3% (w/w).

Adding the β-cyclodextrin/β-cyclodextrin derivative and/or the mangiferin at, or approximately at their solubility limits, reduces the level of unreacted compounds in the mixture after complexation and results in a more efficient process. This is particularly useful for mangiferin, which is an expensive product, and further increases its viability as a cosmetic ingredient.

In an embodiment, the β-cyclodextrin or β-cyclodextrin derivative is β-cyclodextrin.

In an embodiment, the β-cyclodextrin or β-cyclodextrin derivative is a β-cyclodextrin derivative. The β-cyclodextrin derivative can be selected from hydroxypropyl-β-cyclodextrin, sulfobutylether β-cyclodextrin and mono (6-ethylene-diamino-6-deoxy)-β-cyclodextrin.

In an embodiment, the step of allowing the complex to form includes stirring of the solution. Preferably the stirring is continuous. Optionally, the stirring takes place at intervals.

The invention also relates to a method of preparing a skin care composition comprising:
preparing a water phase comprising the additive described above;
preparing an oil phase;
adding the water phase to the oil phase to form an initial water-in-oil emulsion and subsequently an oil-in-water emulsion.

The emulsion may be prepared using an “inversion” method. In this method, a water-in-oil emulsion is created initially after a small amount of the water phase has been added to the oil phase. As further water is added, the emulsion inverts to an oil-in-water emulsion. This method is preferable for achieving a small droplet size. The small droplets are formed at the inversion point, and therefore the water should be added relatively slowly, preferably dropwise, until this point is passed.

In an embodiment, the emulsion is subjected to at least one microfluidisation step.

Microfluidisation is a technique used to reduce particle sizes in emulsions. During the microfluidisation step, high pressures are applied to force a liquid through a chamber of microchannels.

The microfluidisation step may be performed at a pressure of from 5,000 to 15,000 psi.

The emulsion may have a droplet size of from 100-120 nm.

Droplet size may be measured using a laser diffraction particle sizer.

### EXAMPLES

### Materials

Mangiferin (Sigma Aldrich)

β-cyclodextrin (Sigma Aldrich);

Isohexadecane emollient oil (Arlamol HD™ from Croda™);

Polysorbate 60 non-ionic surfactant (Tween 60™ from Croda™);

Polysorbate 61 non-ionic surfactant (Tween 61™ from Croda™);

Sodium stearoyl glutamate ionic surfactant (Eumulgin SG™ from Cornelius (distributed by BASF UK);

PEG-8 solvent (Pluracare E400 SG™ from Cornelius);

Octocrylene organic sunscreen (MfSorb 104™ from Aston Chemicals or Surfacare PCE597™ from Surfachem™);

Avobenzone organic sunscreen (Mfsorb 502™ from Aston Chemicals);

Phenoxyethanol preservative (Preserve PCG 50% phenoxyethanol in Caprylyl glycol from Gracefruit);

Vitamin E antioxidant (from Gracefruit);

Titanium dioxide inorganic sunscreen 40wt % dispersed in water (Solaveil™ CT12W from Croda™)

The following examples are included by way of illustration only, and are not intended to be limiting on the invention, the scope of which is defined by the appended claims.

### Example 1: Cell Viability Assay for Varying Concentrations of Mangiferin Extract

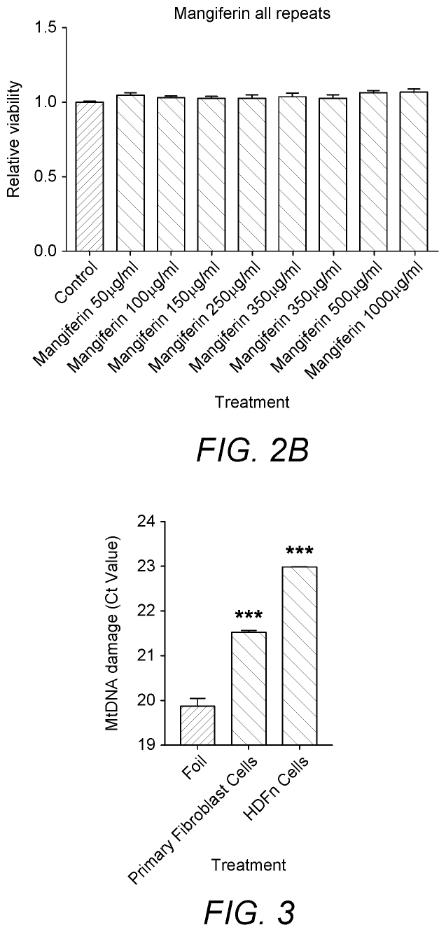

Stock solutions of mangiferin were made by dissolving mangiferin extract in water, and diluting to the required concentration. The stock solutions were then added to HDFn cells at a range of concentrations from 50 μg/ml to 50,000 μg/ml, and incubated for 24 hours under standard culture conditions. Viability was then analysed using an MTS cell proliferation assay, and the results are shown in FIG. 2. The differences in viability between the control (no extract) and the different extract concentrations were compared to determine significance (FIG. 2 shows n=2 biological repeats, of 8 technical repeats each).

The results illustrate cell viability up to concentrations of ^{˜}5,000 μg/ml, above which concentration cell death was observed.

While the results shown are for unfiltered extract with HDFn cells, similar results were observed when the mangiferin was filtered, and also when the mangiferin extract was tested on primary human fibroblast cells grown directly from the skin of donors.

### Example 2: Analysis of mtDNA Damage in Human Skin Fibroblasts and HDFn Cells Following UV Radiation

mtDNA damage in primary human skin fibroblasts and HDFn cells was determined by qPCR in primary human skin cells and in a human dermal cell line (HDFn) following UV irradiation with a Cleo Performance lamp (iSOLde, Germany). mtDNA damage was found to be induced the most in HDFn. As illustrated in FIG. 3, primary fibroblasts showed an increase in mtDNA damage of 1.6 Cts following UV irradiation at 2 standard erythemal doses (SED) compared to the foil-covered control cells. HDFn cells showed a difference in mtDNA damage of 3.1 Cts following irradiation. Since a 1 Ct difference is equivalent to a 2-fold difference in damage, the primary fibroblast cells showed ^{˜}3-fold more damage (2\hat{ }1.6), whereas the HDFn cells showed a much larger increase in damage of ^{˜}8-fold (2\hat{ }3.1). HDFn cells were therefore chosen to assess the protective effect of mangiferin extract in further studies.

### Example 3: Analysis of Protective Effect of Mangiferin Extract on mtDNA Damage in HDFn Cells

1,000 μg/ml solutions of mangiferin extract were prepared as outlined in Example 1 above. 3 ml of the mangiferin solution was added to a 45 mm diameter quartz glass petri dish. Quartz was chosen as it is known to permit both UVA and UVB rays to pass through. The quartz glass dish was then placed on top of a slightly smaller dish containing the cultured HDFn cells. The cells were then irradiated through the quartz glass petri dish, using either a Cleo Performance (iSOLde, Germany) or Aramid B (Cosmedico, Germany) lamp at 2 standard erythemal doses (SED). In order to assess damage caused by irradiation, controls were carried out with water in the quartz glass dish, and by providing non-irradiated cell dishes wrapped in foil to protect against UV-induced DNA damage.

Following irradiation, total DNA was extracted and mtDNA damage analysed using a 1 kb real-time qPCR assay. mtDNA damage is expressed as a Ct value (where a 1 Ct difference is equivalent to a 2-fold difference in damage), and is based on the observation that damaged mtDNA takes longer to be amplified via qPCR. To find the acellular UV transmission of extracts, the dish containing the diluted extract was placed on top of a spectroradiometer and the amount of UV which passes through the dish was measured. All statistical differences were determined using one-way ANOVA with Dunnett's test to compare to a control column (0.05*, P<0.01**, P<0.001***).

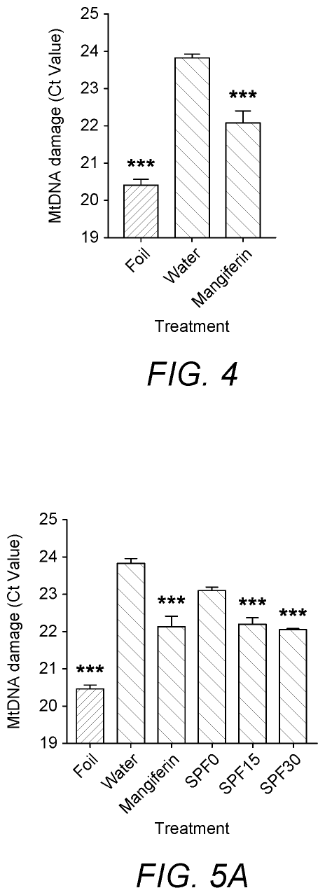

As illustrated in FIG. 4, mangiferin extract showed significant prevention of mtDNA damage. The difference between the foil-covered cells and the water control was 3.1 Cts, or an approximately 8-fold increase in damage. In the presence of mangiferin, this increase in damage was reduced to 1.6 Cts, or an approximately 3-fold increase in damage compared with the foil-wrapped samples (i.e. no irradiation). This study therefore indicates that mangiferin extract may have excellent UV preventative properties.

### Example 4: Comparison of Protective Effect of Mangiferin Extract on mtDNA Damage With That Provided by SPF15 and SPF30 Products

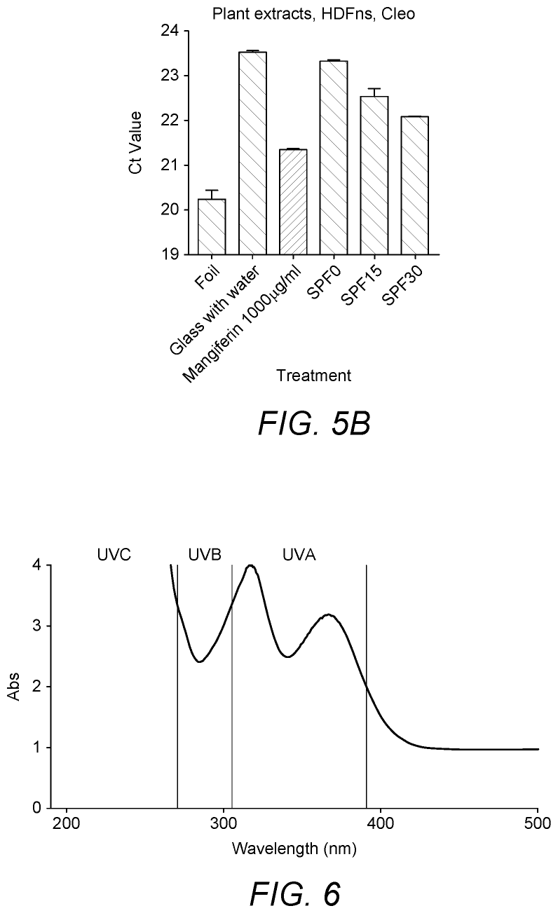

To determine the efficacy of mangiferin extract in comparison with known UV-protection compounds, the experiments of Example 3 were repeated using SPF0, SPF15 and SPF30 cream products. The results of this study are shown in FIG. 5 and suggest that mangiferin extract may provide similar protective effects to known commercial products.

### Example 5: UV Absorbance of Mangiferin Extract

A UV absorbance scan was performed on mangiferin extract at a concentration of 1,000 μg/ml, and the results are shown in FIG. 6. The spectrometer was set up using a quartz cell with a 1.05 ms integration time (the minimum for the spectrometer) and the spectra was based on an average of 100 samples. Mangiferin shows peak absorbance in the UVA region, with absorbance peaks at ^{˜}320 nm and ^{˜}370 nm. This strongly suggests utility in UV protection of human skin, since the majority (96%) of UV rays that reach the earth's surface are within the UVA spectrum, compared with UVB (only 4%).

Example 6: Solubility of Mangiferin

In order to assess the solubility of mangiferin in aqueous solution, 0.18 g of mangiferin was added to 100 mls of deionised water. The solution was heated to 40° C. and stirred at 500 rpm for 4 hours or for 12 hours. The solution was then Buchner filtered, and the mass of the undissolved material weighed. Solubility was calculated based on the difference between the initial mass of mangiferin and that of the undissolved material. The results are shown in Table 1, and indicate that mangiferin is very insoluble in water, even after stirring for 24 hours.

TABLE 1
Solubility of mangiferin

| Stirring | Initial | Mass of | Mass of | Solubility | |
|---|---|---|---|---|---|
| time | mass of | undissolved | mangiferin | of | |
| (40° C., | mangiferin | material | in solution | mangiferin | Precipitate |
| 500 rpm) | (g) | (g) | (g) | (g/L) | formed |
| 4 hours | 0.1826 | 0.1783 | 0.0043 | 0.043 | Very small |
| | | | | | amount of |
| | | | | | yellow |
| | | | | | precipitate |
| | | | | | (<01%) |
| 24 hours | 0.1831 | 0.1692 | 0.0139 | 0.139 | Yellow |
| | | | | | precipitate |
| | | | | | (<0.1%) |

### Example 7: Preparation of Mangiferin-β-Cyclodextrin Complex

A mangiferin-β-cyclodextrin complex was prepared as follows. 1.85 g of β-cyclodextrin was added to 100 ml of deionised water and stirred at room temperature until dissolution of all of the solids had occurred. 0.18 g of mangiferin was added to the β-cyclodextrin solution and the solution was stirred at 500 rpm for 4 hours. The solution was filtered using Buchner filtration. Formation of the mangiferin-β-cyclodextrin complex was confirmed by FTIR.

### Example 8: Solubility of Mangiferin-β-Cyclodextrin Complex

In order to assess the solubility of the mangiferin-β-cyclodextrin complex in aqueous solution, 1.85 g of β-cyclodextrin was dissolved in 100 ml of deionised water at 40° C. ^{˜}0.18 g of mangiferin was added to the β-cyclodextrin solution, and the solution was stirred at 500 rpm for 4 or 24 hours at 40° C. The solution was then Buchner filtered at 40° C. (Examples 1, 2, 6 and 7), hot filtered (Example 3), cold filtered (Example 4), or unfiltered (Example 5). Example 6 used β-cyclodextrin hydrate, and Example 7 studied the effect of using a co-solvent. Solubility was calculated based on the difference between the initial mass of mangiferin and β-cyclodextrin and that of the undissolved material. The results are shown in Table 2.

TABLE 2
Solubility of mangiferin-β-cyclodextrin complex

| | Stirring | | | | | | | |
|---|---|---|---|---|---|---|---|---|
| | time | | | Initial | Mass of | Volume | | |
| | (500 | Stirring | | mass of | residual | of | Solubility | |
| Ex. | rpm) | temp. | Filtration | MGN | MGN | water | of MGN | Precipitate |
| No. | hours | ° C. | temp | (g) | (g) | (ml) | (g/l) | formed |
| 1 | 4 | 40 | 40 | 0.1837 | 0.1064 | 100 | 0.773 | Clear/white |
| | | | | | | | | precipitate (<1%) |
| 2 | 24 | 40 | 40 | 0.1850 | 0.1153 | 100 | 0.697 | Clear/white |
| | | | | | | | | precipitate (<1%) |
| 3 | 4 | 60 | Hot | 0.0912 | 0.0575 | 50 | 0.674 | Clear precipitate |
| | | | | | | | | (<1%) |
| 4 | 4 | 60 | cold | 0.0912 | 0.0503 | 50 | 0.818 | Very narrow |
| | | | | | | | | supernatant |
| | | | | | | | | yellow insoluble |
| | | | | | | | | (<1%) |
| 5 | 4 | 40 | NONE | 0.0695 | n/a | 100 | 0.0695 | Dark solids |
| | | | | | | | | suspended in |
| | | | | | | | | solution |
| | | | | | | | | (impurities) small |
| | | | | | | | | amount of clear |
| | | | | | | | | precipitate (<1%) |
| 6* | 4 | 40 | 40 | 0.1814 | 0.1195 | 100 | 0.619 | Clear precipitate |
| | | | | | | | | (<1%) |
| 7** | 4 | 40 | 40 | 0.1850 | 0.1233 | 100 | 0.617 | None |

*cyclodextrin hydrate used instead of cyclodextrin
**10:90 Ethanol:water used instead of water

These results indicate that the complexed mangiferin is ^{˜}18-fold more soluble than mangiferin on its own over a 4-hour complexation period (i.e. from 0.043 g/l to 0.773 g/l). A ^{˜}5.6 fold increase was observed over the 24 hour solubility of mangiferin (i.e. from 0.139 g/l to 0.773 g/l). An increased complexation time did not result in an increase in solubility. An increase in temperature (i.e. from 40° C. to 60° C.) also did not result in increased solubility (in fact a minor insignificant decrease was observed).

### Example 9: UV Absorbance of Mangiferin-β-Cyclodextrin Complex

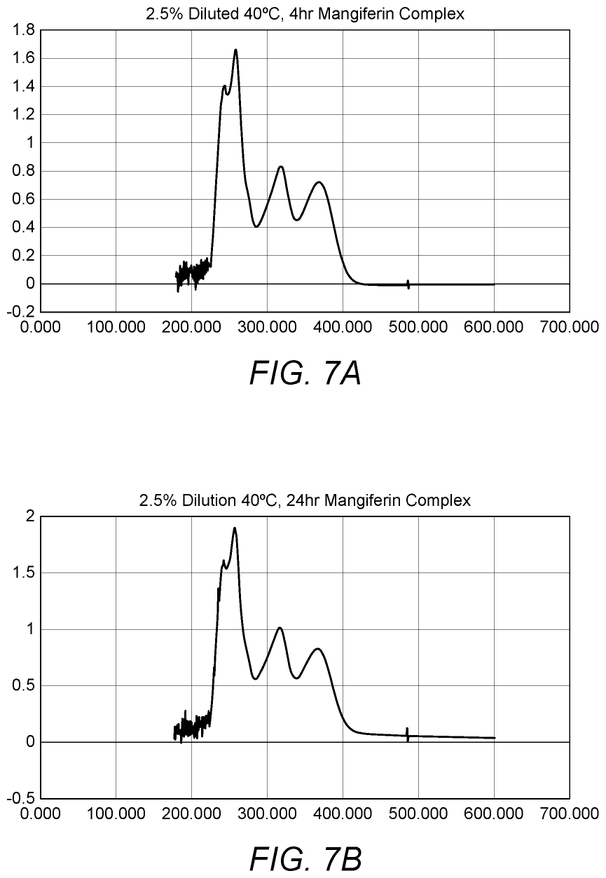

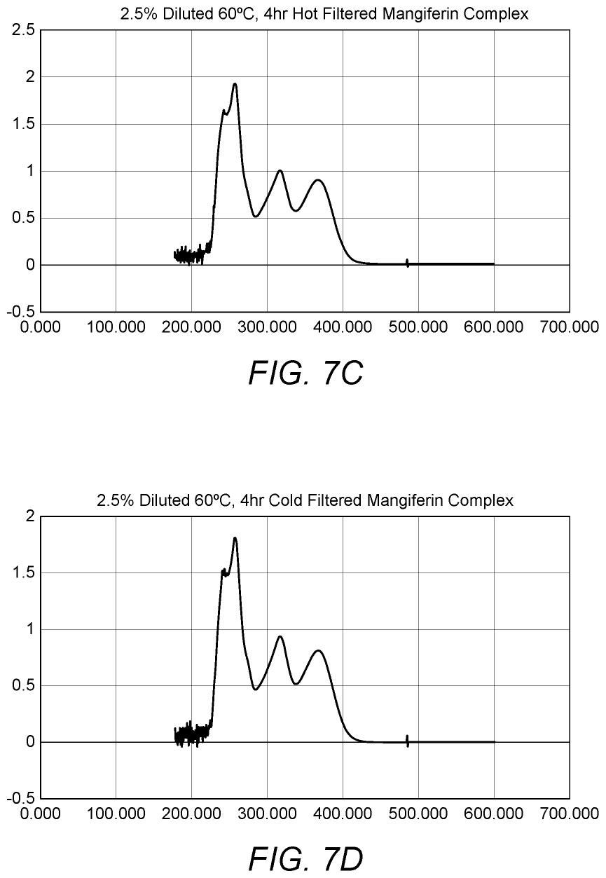

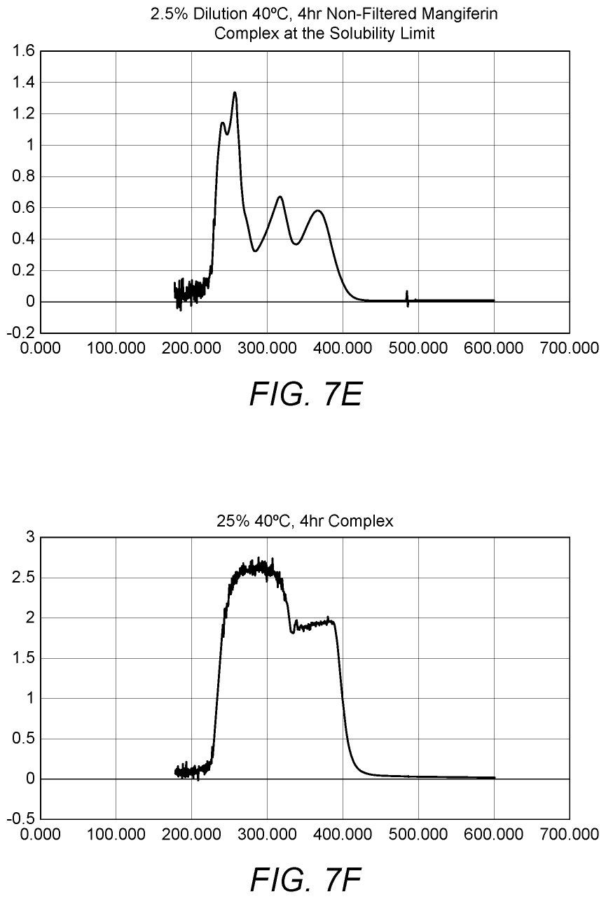

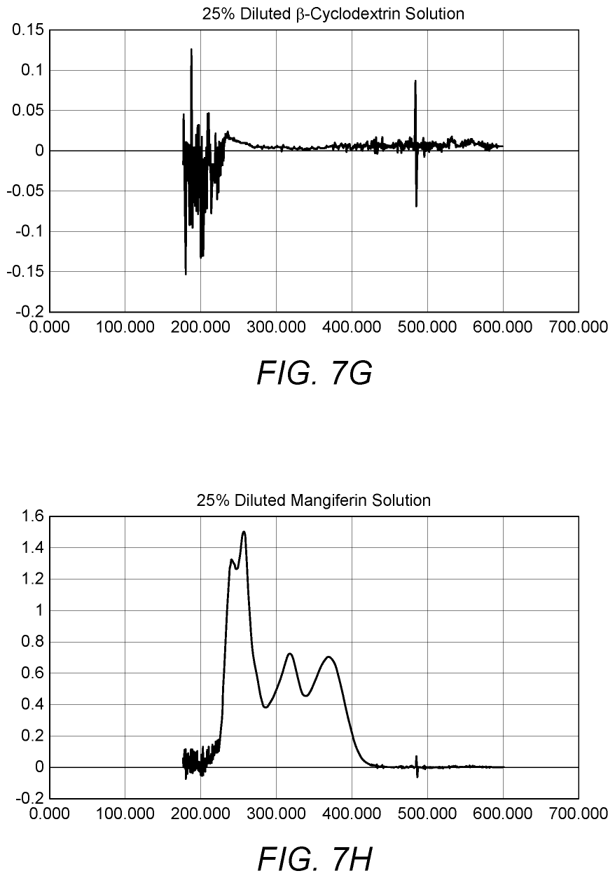

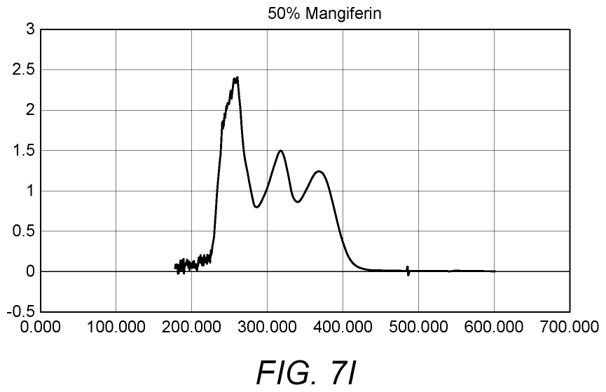

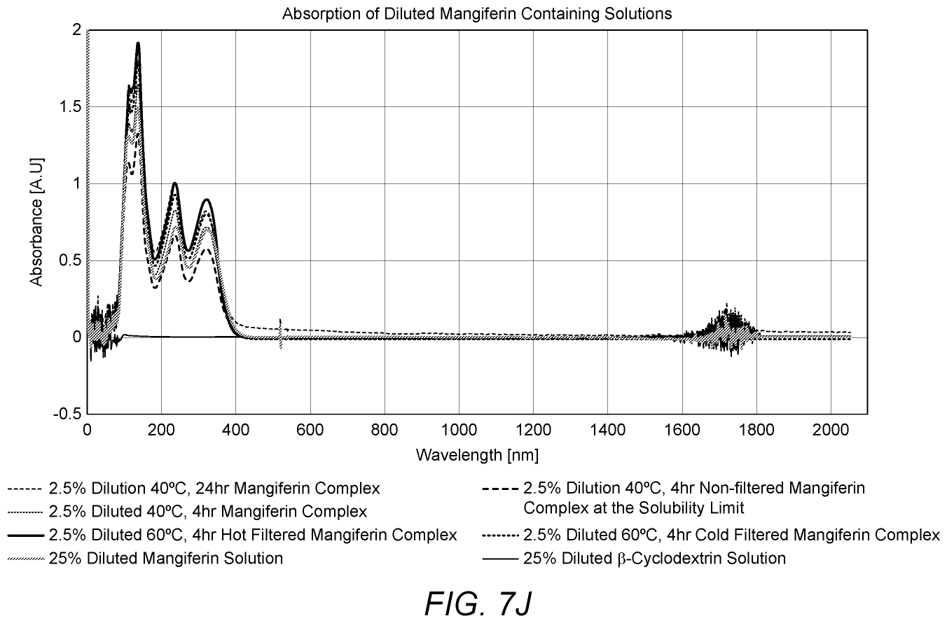

UV absorbance scans were performed on stock solutions of the mangiferin-complexes prepared in example 8 above and the results are shown in FIG. 7. The spectrometer was set up using a quartz cell with a 1.05 ms integration time (the minimum for the spectrometer) and the spectra was based on an average of 100 samples. The mangiferin-only solution required to be diluted to 25% of its original concentration to reach the point at which it no longer saturated the spectrometer, while mangiferin-complex solutions were diluted to 2.5%. However, good absorbance was then observed for all of the samples, with the characteristic peaks at ^{˜}320 nm and ^{˜}370 nm clearly visible.

### Example 10: Preparation of Mangiferin-Containing Sunscreen Formulation

### 10.1 Mangiferin Encapsulated in β-Cyclodextrin

A water bath was set up on a hot plate on a lab jack with a temperature probe controlling the bath temperature to 40° C. A stirrer bar was placed into the water bath and set to 400 rpm.

A 1,000 ml beaker was filled with deionised water (500 ml) and clamped above the water bath. A stirrer bar was added to the beaker. The hotplate and bath were then raised up to the beaker to submerge it to the bath close to the fill line. The stirring speed was kept at 400 rpm. β-cyclodextrin (9.00 g) was added to the beaker, and allowed to fully dissolve.

Once the β-cyclodextrin was fully dissolved, mangiferin (0.36 g) was added to the beaker. The beaker was then capped with aluminium foil and left to stir at 40° C. for 4 hours.

The beaker was then removed from the water bath, decanted into a 500 ml Duran bottle for use in the full formulation sunscreen, and left to cool to room temperature before being stored in the fridge.

### 10.1.1. Creation of the Oil Phase

The following were weighed out into a 500 ml beaker:

Octocrylene (66.75 g), Avobenzone (13.33 g), Isohexadecane (19.73 g), Polysorbate 60 (5.91 g),

Polysorbate 61 (10.52 g), Eumulgin SG (1.04 g), PCG (8.05 g) and Vitamin E (0.48 g).

The Polysorbates needed pre-warming to enable aliquots to be taken from their containers. The beaker was then placed into a stirrer water bath at 50° C. An overhead stirrer was then placed into the beaker and stirred at ca.200 rpm.

### 10.1.2. Creation of the Water Phase

The Mangiferin complex solution was filtered through a Whatman No1 filter using a Buchner filter. Mangiferin complex solution (154.52 g) was weighed out into a 200 ml beaker and PEG-8 (20.02 g) added. The beaker was then placed into a stirred water bath at 50° C. A magnetic stirrer bar was added to the beaker and stirred at 290 rpm.

### 10.1.3. Creation of the Emulsion

The emulsion was created using an ‘inversion’ method, i.e. a water-in-oil emulsion was created initially after a small amount of water had been added to the oil phase, with further water addition the emulsion inverted to an oil-in-water emulsion. The method is preferred over the ‘direct’ emulsion process (adding the oil phase to the water phase) in order to achieve small oil droplets. The small droplets are formed at the inversion point thus the water should be added relatively slowly until this point is passed (often indicated by an increase in the formulation viscosity). Hence the water phase was added to the oil phase (stirred at 433 rpm) in a steady stream of drops using a disposable pipette. After the addition was finished the emulsion was allowed to stir for 10 minutes. The emulsion was then transferred to the Silverson high shear mixer and mixed at 4,440 rpm for 15 minutes. A sample (^{˜}15 ml) was then taken to determine the effect of subsequent micro-fluidisation on particle size.

The emulsion was then transferred to the high pressure homogeniser (Microfluidizer from Microfluidics Corp.) where it passed through the equipment at 10,000 psi. 3 samples of approx. 50 ml were collected before the equipment blocked during collection of the 4th sample. These samples, S1 to S3, were subjected to particle size analysis in Example 11 below. It was visually observed that after being passed through the micro fluidizer the white emulsion of the mangiferin-containing sunscreen formulation gained a blue tinge around the edges of the solution. This was an indication that the microfluidizer had reduced the particle size to be comparable with visible wavelengths.

### 10.2 Water-based Sunscreen Formulation (Mangiferin-Free, Reference Formulation)

A reference formulation, in which the mangiferin complex was replaced with deionised water, was prepared as follows:

### 10.2.1. Oil Phase

The following were weighed out into a 500 ml beaker:

Octocrylene (66.71 g), Avobenzone (13.37 g), Isohexadecane (19.90 g), Polysorbate 60 (6.03 g),

Polysorbate 61 (10.03 g), Eumulgin SG (1.05 g), PCG (7.88 g), and Vitamin E (0.49 g).

### 10.2.2. Water Phase

Water (154.52 g) was weighed out into a 200 ml beaker and PEG-8 (20.03 g) added.

### 10.2.3. Creation of the Emulsion

The same process as described in Example 10.1 above was adopted. In this case no Microfluidizer blockage occurred, enabling 6 samples of approximately 50 ml each to be collected (Samples S5 to S10). Samples S5, S6 and S7 were passed through the microfluidizer a second time at 10,000 psi., and collected as 3 samples (numbered S11 to S13).

### 10.2.4. Addition of TiO₂

Sample 8 from the microfluidizer (50.66 g) was taken and weighed into a clean glass jar. Solaveil™ CT 12 W (5.63 g) was weighed out into the formulated sunscreen. The two were then mixed at 4500 rpm for 2 minutes on the Silverson high shear mixer.

### Example 11: Particle Size Analysis

The particle size of the droplets prepared in Example 10.1.3 (i.e. S1 to S3) was analysed using a Horiba LA950 laser diffraction particle sizer. The refractive index used was 1.564: this is a weighted average of Octocrylene (1.567) and Avobenzone (1.546) based upon their mass within the formulation.

The microfluidisation was shown to reduce particle size to between 100-120 nm. This compares favourably with the average particle size in leading commercial products, which is typically of the order of ca. 130 nm. The reduction in particle size resulted in the edges of the white emulsion having a blue tint to them. Reduced particle size is typically associated with improved sensorial feel and a semi-transparent to transparent look on a user's skin, both of which are highly advantageous properties for sunscreens or cosmetic formulations, and therefore the reduced particle size of the mangiferin-containing sunscreen formulation is highly beneficial.

The particle size analysis was repeated for the water-based sunscreen formulation (mangiferin-free, reference formulation) prepared in Example 10.2 above. The results were similar to those obtained for the mangiferin-containing formulation. The addition of titanium dioxide in Example 10.2.4 did not change the particle size distribution. A second pass on the micro fluidizer did not reduce the droplet size any further than what had already occurred after the first pass.

### Example 12: Stability Testing

Stability testing was done at 40° C. and assessed both visually and instrumentally. Samples were placed into the ‘Turbiscan’ (Formulation TAGS) ageing station at 40° C. A spectrum of the transmitted and backscattered light was taken every 3 hours for 31 days. Some samples were left longer than the test period at 40° C. but no further data was collected. Table 3 below shows the tested samples, the time they were tested for and the results of the test.

TABLE 3
Results of stability testing at 40° C.

| | Time of | |
|---|---|---|
| | Stability | |
| | Test | |
| Sample | (Days) | Visual observations |
| Mangiferin-based sunscreen pre- | 46 | 60% creamed emulsion |
| microfluidiser | | |
| Mangiferin-based sunscreen after | 49 | ca. 2% coalesced oil layer |
| 1 pass on the micro fluidiser | | on top of water |
| Mangiferin-free sunscreen pre- | 32 | 75% creamed emulsion |
| microfluidiser | | |
| Mangiferin-free sunscreen after 1 | 32 | No separation |
| pass on the micro fluidiser | | |
| Mangiferin-free sunscreen after 2 | 32 | A good emulsion but starting |
| passes on the micro fluidiser | | to form a coalesced oil |
| | | droplet on top |
| Mangiferin-free sunscreen after 1 | 32 | A good emulsion but starting |
| pass on the micro fluidizer and | | to form oil droplets on top |
| addition of TiO₂ | | |

The improvement in stability resulting from the microfluidisation was evident from a visual observation, with the larger droplets of the pre-microfluidised samples showing extensive creaming. Thus, the microfluidised samples were shown to exhibit generally good stability after prolonged periods at elevated temperatures. The mangiferin-based sunscreen formulations showed good initial colloidal stability although some coalescence was observed after a month at elevated temperatures.

### Example 11: Seahorse Analyser

The Seahorse XFe96 analyser is utilised to monitor oxygen consumption rate (OCR) and extracellular acidification rate (ECAR) of cells in real time. OCR predominantly represents mitochondrial function with oxygen consumed as the final acceptor of electrons, vital in formation of cellular energy (ATP) via oxidative phosphorylation (Oxphos). ECAR represents the complimentary energy pathway of glycolysis, which has a significantly lower yield of ATP from glucose, in the absence of oxygen.

The aim of this experiment was to test the protective potential of mangiferin-based sunscreen formulations against solar radiation. Human dermal fibroblasts (HDFn) were plated in the Seahorse 96 well culture plates. Cells were irradiated with a solar simulator, delivering UV, visible and infrared light, replicative of the solar spectrum. A dose of 4.32 standard erythemal doses (SED) was given. Formulations in cream form were spread equally across semi-porous tape at a mass to area of 50 mg/cm². Formulations were placed between light source and cells. Following irradiation, cells were bioenergetically analysed using a mitochondrial stress test assay monitored with the Seahorse analyser. The formulations tested were the mangiferin-based sunscreen formulation prepared in Example 10.1 (which was run as two separate tape preparations, as MGN-1 and MGN-2), the water-based sunscreen formulation (mangiferin-free, reference formulation) prepared in Example 10.2 (as AQ-1), and a leading commercial product (LCP-1), which comprised the following ingredients: Aqua/Water, Diisopropyl Sebacate, Alcohol Denat, Glycerin, Dimethicone, Isohexadecane, Butyl Methoxydibenzoylmethane, Octocrylene, Silica, Drometrizole Trisiloxane, Isononyl Isononanoate, Zea Mays Starch/Corn Starch, C12-C15 Alkyl Benzoate, Styrene/Acrylates Copolymer, Ethylhexyl Triazone, PEG-30 Dipolyhydroxystearate, Bis-Ethylhexyloxyphenol Methoxyphenyl Triazine, Isododecane, Phenoxyethanol, Isopropyl Lauroyl Sarcosinate, Terephthalylidene Dicamphor Sulfonic Acid, Silica Silylate, Lauryl PEG/Ppg-18/18 Methicone, PEG-8 Laurate, Caprylyl Glycol, Triethanolamine, Disteardimonium Hectorite, Tocopherol, Disodium EDTA, Dodecene, Propylene Carbonate, Poloxamer 407, Zinc Gluconate, Perlite.

### 11.1 Basal Oxygen Consumption

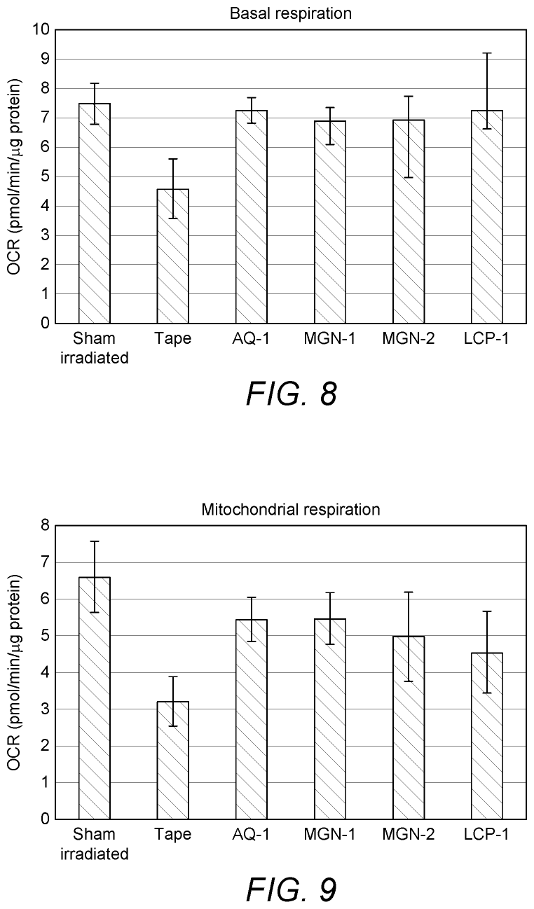

The results of the basal oxygen consumption test are shown in FIG. 8. Whole cell total oxygen consumption is significantly reduced with no protection (tape control) (p=0.0003). All sunscreen formulations tested did not show a reduction in total oxygen consumption, indication solar protection. Data represents a single biological repeat (N=1) with multiple experimental repeats (n=4-6). The mangiferin sunscreen was tested as two separate tape preparations

### 11.2 Mitochondrial Respiration

Oxygen consumption is influenced by multiple cellular processes; however, mitochondria respiration is the predominant consumer. The level of decline in mitochondrial population can therefore be used as a marker of mitochondrial DNA damage. The results shown in FIG. 9 illustrate that all of the tested formulations showed moderate levels of decline. While mitochondrial specific respiration was not significantly reduced by 4.32 SED when mangiferin-based sunscreen was applied for one group (MGN-1), the second group (MGN-2) showed a significant level of mitochondrial OCR decline (p=0.0343). However, the leading commercial product (LCP) also displayed mitochondrial decline with a higher statistical significance value (p=0.0056).

### 11.3 Glycolytic Index

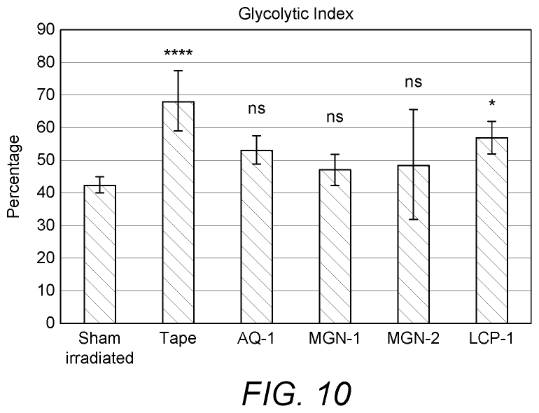

Glycolytic index represents the percentage of energy demands met by glycolysis for cells. As mitochondria are damaged, cells become reliant on glycolysis to meet energy demands, and therefore an increase in glycolysis can be used as an indicator of mitochondrial damage. The results of this experiment are shown in FIG. 10. The control (tape only) showed the most damage to mitochondria as cells become more glycolytic (p=0.0001). The only population with protection to show significant increase in glycolysis was the leading commercial product (p=0.0326). All other tested formulations suggest they have significantly blocked solar radiation to prevent mitochondrial damage and halt reliance on glycolysis.

The results of the various tests set out above confirm mangiferin as a useful extract in UV protection for human skin. The results further, and advantageously demonstrate that complexes of mangiferin with β-cyclodextrin or a β-cyclodextrin derivative protect against mtDNA damage, and as such, can be used as additives in skin care products, to protect against UV radiation and to provide positive effects such as anti-aging effects associated with a reduction or prevention of mtDNA damage. The complexation of the mangiferin with the β-cyclodextrin/β-cyclodextrin derivative, beneficially increases the availability of the mangiferin in the aqueous composition/aqueous phase, which retaining the mtDNA damage-preventing effects. Comparable particles sizes (i.e. 100-120 nm) compared with leading competitor products (typically ^{˜}130 nm), suggest ease of application to the skin with transparent/near transparent properties. These properties confirm that mangiferin can be used in the preparation of an economically-viable skin care product, such as a suncream, or other cosmetic product in which mtDNA damage prevention is desirable.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A topical composition comprising a compound having the following general formula I: 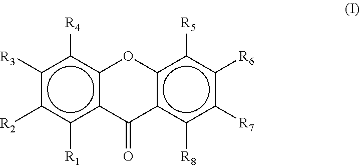
wherein each of R₁, R₂, R₃, R₄, R₅, R₆, R₇, R₈ is selected from the group consisting of —H, —OH and a glucosyl radical, or an ester or pharmaceutically acceptable salt thereof, for the prevention of mitochondrial DNA damage.

2. A topical composition as in claim 1 wherein the compound is mangiferin, having the formula: 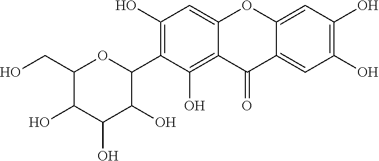 [SMILES: O=c1c2cc(O)c(O)cc2oc2cc(O)c(C3OC(CO)C(O)C(O)C3O)c(O)c12]
or isomers or derivatives or an ester thereof or one of its pharmaceutically acceptable salts.

3. (canceled)

4. (canceled)

5. (canceled)

6. A topical composition as in claim 1 wherein the composition is a pharmaceutical composition.

7. A topical composition as in claim 1 wherein the composition is a cosmetic composition.

8. A topical composition as in claim 1 wherein the composition is in the form of a cream, oil, ointment or lotion.

9. An additive for a skin care composition, wherein the additive comprises a complex of mangiferin with β-cyclodextrin or a β-cyclodextrin derivative.

10. An additive as claimed in claim 9, wherein the β-cyclodextrin derivative is selected from hydroxypropyl-β-cyclodextrin, sulfobutylether β-cyclodextrin and mono (6-ethylene-diamino-6-deoxy)-β-cyclodextrin.

11. An additive as claimed in claim 9, wherein the mangiferin-βcyclodextrin complex has a stoichiometric ratio of 1:1.

12. An additive as claimed in claim 9, for the prevention of mitochondrial DNA damage.

13. A skin care composition comprising the additive of claim 9.

14. A skin care composition as claimed in claim 13, wherein the additive is included in the composition such that the concentration of the mangiferin in the composition is from 1 to 10% (w/w).

15. A skin care composition as claimed in claim 13, wherein the composition comprises the mangiferin-βcyclodextrin complex in an aqueous phase.

16. A skin care composition as claimed in claim 13, wherein the composition is in the form of an emulsion.

17. A skin care composition as claimed in claim 13, wherein the composition further comprises at least one additional ingredient selected from an organic UV filter, an inorganic UV filter, an emulsifier, a suspending agent, a film former and a skin conditioner.

18. A skin care composition as claimed in claim 13, wherein the skin care composition is in the form of a cream, lotion, oil, ointment or spray.

19. A method of preparing an additive as claimed in claim 9, the method comprising:
preparing a solution of β-cyclodextrin or a β-cyclodextrin derivative in water;
adding mangiferin to the solution;
allowing the complex to form at a temperature between 30° C. and 50° C. for between 3 to 6 hours; and
removing solids by filtration.

20. A method as claimed in claim 19, wherein the β-cyclodextrin or β-cyclodextrin derivative is added at a concentration of from 1 to 3% (w/w).

21. A method as claimed in claim 19, wherein the mangiferin is added at a concentration of from 0.1 to 0.3%.

22. A method as claimed in claim 19, wherein the β-cyclodextrin or β-cyclodextrin derivative is β-cyclodextrin.

23. A method as claimed in claim 19, wherein the β-cyclodextrin or β-cyclodextrin derivative is a β-cyclodextrin derivative selected from hydroxypropyl-β-cyclodextrin, sulfobutylether β-cyclodextrin and mono (6-ethylene-diamino-6-deoxy)-β-cyclodextrin.

25. A method of preparing a skin care composition comprising:
preparing a water phase comprising the additive of claim 9;
preparing an oil phase;
adding the water phase to the oil phase to form an initial water-in-oil emulsion and subsequently an oil-in-water emulsion.

25. The method of claim 24, comprising subjecting the emulsion to at least one microfluidisation step.

26. A method for preventing mitochondrial DNA damage in mammals, comprising the steps of: administering a topical composition comprising a compound having the following general formula I: 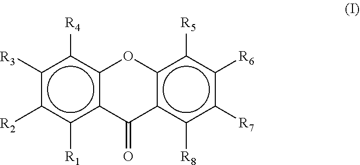
wherein each of R₁, R₂, R₃, R₄, R₅, R₆, R₇, R₈ is selected from the group consisting of —H, —OH and a glucosyl radical, or of one of the esters or its pharmaceutically acceptable salts.

27. A method for preventing mitochondrial DNA damage as in claim 26 wherein the compound is mangiferin, having the formula: 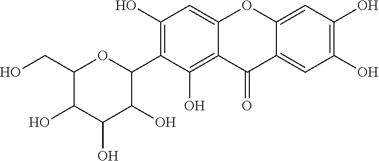 [SMILES: O=c1c2cc(O)c(O)cc2oc2cc(O)c(C3OC(CO)C(O)C(O)C3O)c(O)c12]
or isomers or derivatives or an ester thereof or one of its pharmaceutically acceptable salts.

28. A topical composition as in claim 2, wherein the compound is a naturally-occurring mangiferin extract.

29. A topical composition as in claim 28 wherein the mangiferin extract is present at an effective concentration for protecting against DNA damage.

30. A topical composition as in claim 29 wherein the mangiferin extract is present at an effective concentration of 1000 μg/ml or greater.

31. A topical composition as in claim 30 wherein the mangiferin extract is present at an effective concentration of between 1000 μg/ml and 5000 μg/ml.
